(19) United States
(12) Patent Application Publication
(10) Pub. No.: US 2002/0081289 A1
(43) Pub. Date: Jun. 27, 2002

(54) Ophthalmic medicament

(76) Inventor: Tobias Neuhann, Munich (DE)

Correspondence Address: OSTROLENK FABER GERB & SOFFEN, 1180 AVENUE OF THE AMERICAS, NEW YORK, NY, 100368403

(21) Appl. No.: 09/953,016

(22) Filed: Sep. 14, 2001

(30) Foreign Application Priority Data
Sep. 14, 2000 (DE) 10045343.0

(51) Int. Cl. A01N 1/02; A61K 38/44; A61K 31/737; A61K 31/7076; A61K 31/7036; A61K 31/70; A61K 31/52

(52) U.S. Cl. 424/94.4; 514/23; 514/54; 435/1.1; 514/263.4; 514/37; 514/46

(57) ABSTRACT

According to the invention, a novel ophthalmic medicament is made available, which contains in aqueous solution or suspension at least one carbohydrate, at least one amino acid, at least one electrolyte, a chondroitin sulfate and optionally further customary excipients.

The invention relates to a novel ophthalmic medicament which can be employed in a large number of eye diseases and in particular in accompaniment to corneal transplantation and in refractive corneal surgery.

Although novel ophthalmic medicaments are continually being developed and also just recently a number of highly active ophthalmic medicaments have been developed for all sorts of eye diseases, for a number of eye diseases there are still no satisfactory medicaments or a satisfactory action can only be achieved using steroidal medicaments which contain such a high dose of steroids that they exhibit a number of side effects, for example an intraocular pressure increase.

Especially recently, refractive corneal surgery such as, in particular, Lasik has been used for the elimination of amblyopia. There are presently no satisfactory opthalmic medicaments for concomitant treatment and in particular for after-treatment in the case of refractive corneal surgery.

On the other hand, agents are known in which removed human corneas can be preserved over a relatively long period of time. On its front surface, the human cornea has a multilayered, rapidly renewing, not very sensitive epithelium and on its back or inner side a single-layered, very sensitive endothelium. In a modern preservative solution, such as is commercially available, for example, under the name Optisol®, it has now become possible to store human corneas for several weeks such that they remain suitable for transplantation. By this means, it became possible to install corneal banks and thus to coordinate organizationally the globally continual need for transplantation material. Using preservative solutions of this type, on the one hand the autolysis (decomposition after death) is considerably delayed and on the other hand the death of the sensitive endothelium is markedly slowed down.

Preservative solutions of this type as a rule contain sugars, in particular dextran or glucose, vitamins, essential and nonessential amino acids, electrolytes, iron compounds, a buffer system, such as chondroitin sulfate, L-hydroxyproline, adenosine, inosine, adenine, lactoferrin and lactoperoxidase and also one or more antibiotics, e.g gentamycin and streptomycin. As a buffer, HEPES, i.e. N-2-hydroxyethyl piperazine-N-2-ethanesulfonic acid, is frequently employed.

It is an object of the invention to make available novel opthalmic medicaments which are free of steroids or only contain the steroids in very low amounts and which are extremely active in a number of eye diseases and in particular in the concomitant treatment of a refractive corneal surgery.

This object is achieved by the subject of the patent claims.

The achievement of the object is based on the surprising finding that certain aqueous solutions or suspensions, such as are already known as preservative solutions for removed human corneas, show excellent activity in the case of medicinal use as ophthalmic medicaments.

The ophthalmic medicaments according to the invention contain at least one carbohydrate, in particular glucose and/or dextran, one or more essential or nonessential amino acids and an electrolyte which preferably consists of two or more inorganic salts. The agents according to the invention moreover in general also additionally contain at least one buffer system, HEPES (N-2-hydroxyethylpiperazine-N-2-ethanesulfonic acid) being preferred, and at least one antibiotic, in particular gentamycin and/or streptomycin. The agents according to the invention also preferably contain one or more agents selected from iron compounds, L-hydroxyproline, precursors for adenosine triphosphate, in particular adenosine, inosine and adenine, proteins such as lactoferrin and enzymes such as lactoperoxidase. Further customary excipients can also optionally be present in the agents according to the invention.

An inorganic salt or a mixture of two or more inorganic salts, in particular of three or more inorganic salts, is preferably employed as an electrolyte. Suitable inorganic salts which can be used individually or as a mixture are, for example, magnesium salts, potassium salts, selenium salts, sodium salts etc., in particular, for example, calcium chloride, potassium chloride, magnesium sulfate, sodium chloride, sodium hydrogencarbonate, sodium dihydrogenphosphate, iron nitrate, etc. Suitable vitamins are, for example, vitamin B12, biotin, vitamin B6 etc. The agents preferably contain a mixture of two or more, in particular three or more, more highly preferably seven or more, essential and/or nonessential amino acids. As suitable amino acids, all amino acids which are pharmacologically acceptable in the concentrations used can be used, for example L-alanine, L-arginine, L-asparagine, L-aspartate, L-cysteine, L-glutamic acid, L-glutamine, L-histidine, L-isoleucine, L-leucine, L-lysine, L-methionine, L-phenylalanine, L-proline, L-serine, L-thyrosine, L-tryptophan, L-threonine, L-valine, glycine etc. The mixing ratios are not critical. One or more antibiotics are preferably found in the compositions according to the invention, for example selected from gentamycin, penicillin G and streptomycin, gentamycin and streptomycin, in particular as a mixture, being particularly preferred.

The ophthalmic medicaments according to the invention are free of steroids having a hormone action. However, compounds having a steroid structure such as cholesterol can be present in small amounts. The customary side effects of steroidal ophthalmic medicaments, such as intraocular pressure increase, therefore do not occur.

An aqueous solution or suspension is particularly preferably employed according to the invention as an ophthalmic medicament, as it is already known as a storage solution for human corneal transplants and is commercially available.

In this context, for example, storage solutions may be mentioned such as are marketed under the name POLYSOL® by Polytech Ophthalmologie GmbH and are described in publicly accessible use information and have the following composition:

glucose

sodium pyruvate (1 mM)

essential and nonessential amino acids (1.74 g/l)

vitamins (B12, biotin, B6 . . . )

electrolytes (ca., Mg, K, Se . . . )

gentamycin, penicillin G, streptomycin

chondroitin sulfate (2%)

lactoferrin

lactoperoxidase

HEPES and bicarbonate buffer

Phenol Red

pH 7.4

Osmolarity 320 mOsm/kg of H₂O.

Further examples which can be mentioned are also formulations such as are obtainable under the name Likorol® from Laboratoires Opsia-Pharma France. The Likorol® solution has essentially the same composition as the Polysol® product.

The products Inosol and Exosol, which can likewise be employed according to the invention, are likewise obtainable from Opsia-Pharma. The composition of the Inosol solution is as follows:

cellular culture medium (IMDM)

fetal calf serum

glucose

sodium pyruvate (1 mM)

essential and nonessential amino acids (1.74 g/l)

vitamins (B12, biotin, folic acid)

electrolyte (Ca⁺⁺, Mg⁺⁺, K⁺, Se⁺ . . .)

penicillin, streptomycin, amphotericin B

antioxidants

HEPES and bicarbonate buffer

Phenol Red

pH=7.4

Osmolarity=320 mOsm/kg of H₂O

The Exosol solution corresponds to the Inosol solution, only it additionally contains dextran.

The storage solution for corneas commercially obtainable under the name Optisol, which is described, for example, in the American Journal of Ophthalmology 114, 345-356, Sept. 1992, is particularly preferred. Reference is made in this respect to the contents of this publication, in particular to the exact composition of the Optisol® solutions. Optisol® storage solutions for corneas are also described in the publication Arch Ophthalmol—vol. 109, Jun. 1991, 864-868, to which reference is likewise made.

For a person skilled in the art, it is easily possible to replace individual ingredients of the commercially obtainable preservative solutions for removed human corneas by equivalent constituents and, of course, solutions of this type are also preferred according to the invention.

The dosage of the ophthalmic medicaments according to the invention depends on the indication, the age and condition of the patient etc. and can be determined without difficulties by the treating physician. If, for example, the preferred Optisol solution is employed as an ophthalmic medicament for the aftertreatment of refractive corneal surgery by means of Lasik, one drop of the solution in each case are administered three to four times daily to each eye to be treated. Corresponding dosages are also preferred for the other indications with the exception of the use as a rinsing solution in cataract surgery, in which, of course, considerably more of the solution according to the invention has to be employed, which, however, is known to the person skilled in the art.

The ophthalmic medicaments according to the invention can be employed in many different ways. They have proved particularly advantageous as concomitant medication in refractive corneal surgery, in particular in Lasik, but also in PRK and PTK. When using the ophthalmic medicament according to the invention, the corneas, which are frequently in a poor condition after excimer laser surgery, remain clear or are lightened and the healing of small epithelial injuries is favored. Since the ophthalmic medicament according to the invention contains no steroids, no side effects occur which customarily occur in the case of steroidal medicaments, such as, for example, an intraocular pressure increase.

A further preferred use of the ophthalmic medicament according to the invention is as a rinsing solution in cataract surgery or other intraocular interventions. The ophthalmic medicament according to the invention is also equivalent or even superior to customary steroidal agents as an aftertreatment agent in these interventions.

A further area of use of the medicament according to the invention is in corneal transplantation, where the agents can be employed both intraoperatively for the positioning of the anterior chamber, for the clearing of the transplant and for the reduction of rejections, but in particular is suitable postoperatively as eye drops.

Finally, the ophthalmic medicament according to the invention can preferably be employed as a therapeutic in the following indications:

dry eye (somewhat oily)

superficial punctate keratitis

corneal erosion

contact lens wearing problems

incipient corneal edema in cornea guttata (inherited corneal endothelial disease, which is clinically relevant only at a relatively old age, which can cause great problems, particularly in deferred cataract surgery).

The ophthalmic medicament according to the invention induces no intraocular pressure increase and is so well tolerated on the part of the lens epithelium that the crystalline lens remains clear.

The following examples illustrate the invention.

### EXAMPLE 1

A fresh corneal transplantation had become necessary in an 81 year-old female patient because of clouding of a first corneal transplant after six years. The cause of the clouding was not clear, since the objective data of the first transplant were good. After the first implant, an in-patient stay after the operation of 17 days was necessary, i.e. the postoperative healing took place slowly.

In the fresh corneal transplantation, a postoperative therapy was carried out in which Optisol® AT was used as an ophthalmic medicament additionally to a routinely used steroidal medicament (1% strength prednisolone acetate). The administration of one drop in each case was carried out 3 to 4 times daily for 1 week. The patient was already discharged on the third postoperative day, the transplant was surprisingly clear and the new corneal epithelium had already completely regrown. This is unusually quick, in particular with respect to the age of the patient and for rekeratoplastic surgery.

### EXAMPLE 2

A Lasik treatment was carried out on a male patient having the following refraction data:

right eye: −3.75 0.00 25°=0.8/

left eye: −2.25 −2.00 158°=0.7.

After the Lasik treatment the following data resulted:

right eye: 0.00 −0.25 175°=1.6/

left eye: +0.25 −0.25 158°=1.6.

The aftertreatment was again carried out using Optisol® AT for one week with the administration of one drop 3 to 4 times daily.

In comparison to the customary treatments with BSS (“balanced salt solution”) both the pain postoperatively and the biomicroscopic split lamp result were improved to such an extent on the next and on the following days that it was no longer discernible whether the patient had been treated. The biomicroscopic result was better than that which can be achieved, as a rule, by administration of steroids and relatively high-dose antibiotics during the postoperative phase.

### EXAMPLE 3

All sorts of therapies had remained without success in a male patient who suffered from Fuchs' endotheliopathy, i.e., for example, treatment with steroidal eye drops, dehydrating eye drops, eye drops based on hydroxypropylmethylcellulose (tear replacement solutions) and with therapeutic contact lenses. Optisol was administered to the patient in a dosage of in each case one drop 3 to 4 times per day. The visual acuity had already increased from 0.12 to 0.4 within the second day of treatment and the patient was pain-free. Side effects did not occur, and it was possible to move an already planned corneal operation to an undetermined time.

1. An ophthalmic medicament, which comprises in aqueous solution or suspension at least one carbohydrate, at least one amino acid, at least one electrolyte, chondroitin sulfate and optionally further customary excipients.

2. The ophthalmic medicament as claimed in claim 1 or 2, wherein it furthermore contains one or more substances selected from one or more vitamins, one or more iron compounds, L-hydroxyproline, adenosine, inosine, adenine, lactoferrin, lactoperoxidase, one or more antibiotics and a buffer system.

3. The ophthalmic medicament according to one of the preceding claims, wherein the carbohydrate is glucose and/or dextran.

4. The ophthalmic medicament as claimed in one of the preceding claims, wherein the antibiotic contained is gentamycin and/or streptomycin.

5. The ophthalmic medicament as claimed in claim 1, which is a preservative solution for removed human corneas.

6. The ophthalmic medicament as claimed in one of claims 1-5, which contains in aqueous solution or suspension:
glucose and/or dextran
sodium pyruvate
a mixture of three or more essential and/or nonessential amino acids
one or more vitamins
a mixture of three or more inorganic salts as electrolyte
at least one compound selected from gentamycin, penicillin G and streptomycin
a chondroitin sulfate
lactoferrin
lactoperoxidase
buffer
indicator

7. The use of a mixture of least one carbohydrate, at least one amino acid, at least one electrolyte, chondroitin sulfate and optionally further customary excipients for the treatment of ophthalmic diseases.

8. The use as claimed in claim 7, wherein the mixture furthermore additionally contains one or more substances selected from one or more vitamins, one or more iron compounds, L-hydroxyproline, adenosine, inosine, adenine, lactoferrin, lactoperoxidase, one or more antibiotics and a buffer system.

9. The use as claimed in claim 7 or 8 for aftertreatment in refractive corneal surgery.

10. The use as claimed in claim 9, wherein the refractive corneal surgery is Lasik, PRK and/or PTK.

11. The use as claimed in claim 7 or 8 as a rinsing solution in cataract surgery.

12. The use as claimed in claim 7 or 8 for concomitant treatment and for aftertreatment in corneal transplantation.

13. The use as claimed in claim 7 or 8 for the treatment of dry eye, superficial punctate keratitis, corneal erosion, contact lens wearing problems and/or incipient corneal edema in cornea guttata.
